(19) United States
(12) Patent Application Publication
(10) Pub. No.: US 2006/0263325 A1
(43) Pub. Date: Nov. 23, 2006

(54) Compositions comprising soy products and organic salts of certain metals

(76) Inventors: Muriel Morelli, Acquiny (FR); Nathalie Quiry, Val De Reuil (FR); Franck Bardin, St. Pierre Des Elbeuf (FR); Sophie Rouet, Val De Reuil (FR); Thierry Oddos, Meudon (FR)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/074,281

(22) Filed: Mar. 7, 2005

(30) Foreign Application Priority Data
Sep. 7, 2002 (EP) 02078647.1

(51) Int. Cl. A61K 8/97; A61K 33/34; A61K 33/06; A61K 33/32; A61K 36/48

(52) U.S. Cl. 424/74; 424/638; 424/641; 424/757; 424/682

(57) ABSTRACT

This invention relates to compositions comprising soy products and one ore more organic salts selected from zinc, magnesium and copper. It further relates to the use of such compositions in topical formulations, in particular in formulations for increasing skin firmness and elasticity, even tone and texture.

### BRIEF DESCRIPTION OF THE INVENTION

This invention relates to compositions comprising soy products and one ore more organic salts selected from zinc, magnesium and copper. It further relates to the use of such compositions in topical formulations, in particular in formulations for increasing skin firmness and elasticity, even tone and texture.

### BACKGROUND OF THE INVENTION

Skin is composed of three integrated layers: the epidermis, the dermis and the hypodermis. The dermis consists mostly of collagen, which originates from cells called fibroblasts, elastin structural glycoproteins and proteoglycans. Collagen fibers form a supporting mesh responsible for the skin's mechanical characteristics such a strength, texture and resilience. Other cells such as macrophages and leukocytes are also present in the dermis layer.

The hypodermis, joined to the bottom of the dermis, is the deepest layer of the skin. It contains “adipocytes” which store lipids for the subcutaneous tissue to make a fatty layer that protects muscles, bones and inner organs against shocks, and acts as an insulator and source of energy during lean times.

As a first sign of aging, skin becomes less elastic and develops fine lines and wrinkles, which result from the deterioration of the dermis layer. Indeed the skin's ability to replace damaged collagen diminishes and more caps and irregularities develop in the collagen network. This goes along with the appearance of pigment marks, skin thinning and skin sagging. Many factors contribute to skin aging. These include sun exposure, free radicals, some age-related hormonal changes, and smoking.

A number of treatments have been developed that have proved out to be more or less effective in combating the effects of skin aging. These include applying cosmetic products which contain vitamins or vitamin derivatives, in particular vitamin A or its derivatives, such as alpha-hydroxy acids or retinoids, vitamin C, or plant extracts. A particular pathway used in the treatment of the effects of skin aging is by stimulation of dermal human fibroblasts and collagen formation. Agents possessing these properties for example are L-ascorbic acid and in particular retinol.

Another class of products that are used against the effects of skin aging but also have other beneficial effects on the skin are soy-derived products. Soy products and in particular non-denatured soy products are known to retard hair growth as is described in EP-A-1074240. The latter describes compositions and methods for delaying hair growth, reducing hair follicle and hair shaft size and hair shaft pigmentation by topically applying to the skin a composition comprising legume extracts including soymilk.

WO 01/34099 describes the use of non-denatured soy product containing compositions for depigmentation, evening out skin tone and skin texture, skin firming and care of the skin. U.S. Pat. No. 6,555,143 discloses compositions and methods that relate to legume products and in particular to soy products for regulating firmness of the skin, hair or nails; cleansing the skin, hair or nails; reducing and/or delaying hair or nail growth; and a number of other useful applications. EP-A-1236465 describes legume products having trypsin inhibitory activity, in particular soy products, having reduced microbial content and the use thereof in compositions for application on the skin, nails and hair.

Metals salts of magnesium, zinc or copper have been described to possess beneficial effects on cell metabolism and on extracellular matrix synthesis in dermal fibroblasts.

Although existing products for improving the appearance of the skin and/or for combating the effects of skin aging have been applied with varying degrees of success, there nevertheless remains room for improvement. In particular there still is a need for new formulations that are more effective in combating the effects of skin aging and/or that improve the appearance of the skin. Providing compositions that possess some or several of these effects or properties is an object of this invention.

### SUMMARY OF THE INVENTION

The present invention is directed to a composition comprising a soy product and at least one topically acceptable organic salt selected from the group consisting of zinc, magnesium and copper. The soy product in particular can be a non-denatured soy product, e.g. non-denatured soymilk or powder.

Particular skin acceptable non-toxic organic salts of zinc are salts derived from naturally occurring amino acids or from hydroxyalkyl acids, the latter in particular being sugar-derived acids. Preferred organic salts are the salts of gluconic acid and aspartic acid.

The compositions of the invention may contain two or three organic salts having different metals selected from the metals mentioned herein. The compositions in particular may contain salts of as well zinc, copper and magnesium.

The invention further is concerned with a topical formulation comprising a composition as defined herein and further ingredients. The topical formulation can be for dermatological use but in particular is for cosmetic use.

In another aspect the invention provides the use of a composition as defined herein for manufacturing a topical or in particular a cosmetic formulation. The topical or cosmetic formulations in particular are useful for increasing skin firmness and elasticity, even tone and texture, to combat and treat the effects of skin aging, to prevent and treat sun-induced damage and acne.

In a further aspect the invention provides the use, and in particular the cosmetic use, of a composition as defined herein, or of a topical formulation as defined herein, for increasing skin firmness and elasticity, even tone and texture, to combat and treat the effects of skin aging, to prevent and treat sun-induced damage and acne.

Or, alternatively, the invention concerns a method, and in particular a cosmetic method of combating or treating the effects of skin aging and/or improving the appearance of the skin, including increasing skin firmness and elasticity, evening tone and texture, preventing and treating sun-induced damage and acne, controlling or reducing skin pigmentation, retarding hair growth, which method or cosmetic method comprises applying to the affected skin area an amount of a composition or a topical formulation as defined herein, said amount being effective to treat said effects of skin aging and/or improve the appearance of the skin.

Still another aspect of this invention comprises a cosmetic method for the improvement of the external appearance of an individual, said method comprising applying a composition or a topical composition as defined herein to affected skin areas.

### DETAILED DESCRIPTION OF THE INVENTION

The compositions of the present invention contain soy products that may be in the form of a fluid (e.g., soymilk) or a solid (e.g., a soybean powder or soymilk powder). What is meant by “soy product” is a substance derived from the soybean, containing the ingredients naturally found in soybeans, at the relative concentrations as found in the beans. In preferred embodiments, the soy product is a non-denatured soy product. The latter is a soy product which has been obtained by processes that leave the active proteins intact by carefully controlling the process parameters such as the temperature, the extraction media. This can be measured, for example, by the presence of intact soybean trypsin inhibitor (STI) protein.

In another embodiment, the soy product is soymilk. One way to make soymilk is to soak the soybeans in deionized or purified water for several hours, and grind them after they were fully hydrated, with the addition of small quantities of water. (The grinding process allows the soybean milk to be extracted). After collection, the soybean milk may be filtered to remove any residual parts of the bean husk. The soymilk used in this invention can be fresh soymilk as described above, or may be made from soybean powder and water. The soybean powder is milled from soybeans and may also be lyophilized, spray dried, or freeze-dried and the resulting soymilk may or may not be filtered. Such prepared soymilk may have from about 1 to about 90% by weight dry soybean powder. Another example is the use of soymilk powder, made from lyophilized, spray dried or freeze-dried soymilk, with the addition of water and finished with or without filtration or homogenization.

Other methods of soybean extraction could also be used to create the active ingredients used in this invention. For example, but not limited to, the active ingredients could be extracted from ground soybeans using ethanol/water mixtures, followed by the removal of the ethanol from the extract, in such ways that the protease inhibitory activity of the soybean will be retained.

The compositions of the present invention may contain from about 0.1% to about 99% or from about 1% to about 99%, by weight, of the soy product. For example, when a liquid soy product (e.g., soymilk) is used, the composition may contain from about 50% to about 99%, by weight, (e.g., from about 70% to about 99%) of the liquid soy product. For example, when a solid soy product (e.g., soybean powder or soymilk powder) is used, the composition may contain from about 0. 1% to about 50%, by weight (e.g., from about 1% to about 30%, by weight) of the solid soy product. Compositions, which comprise solid soy products may also comprise water (e.g., distilled water or water contained within soymilk) to form a liquid base to the composition (e.g., to form a cream, lotion or gel). Such composition may comprise from about 50% to about 98% by weight (e.g., from about 70% to about 98%, by weight) of water.

The soy products useful in this invention may be produced from all soybean species, regardless of their geographic origin, sun exposure, harvest time and the like. However, specific strains, geographic origins or growth conditions might be preferred.

For example, but not limiting to, soybean strains particularly rich in their trypsin inhibitor (e.g. STI, LTI, BBI) content or growth conditions that result in trypsin inhibitor enrichment in the bean, may be preferred. It should be noted that the soy products useful in the compositions of this invention may have a distinctive odor, which may be tolerable in some cultures, but is undesired in others. If necessary, the odor of the compositions of this invention may be reduced by using soybean products derived from specific strains of soybeans known to produce reduced-odor, including, but not limited to, lipoxygenase-2-deficient beans and those having modified sugar profile, and the like. A process to reduce oxygen levels in the formulation may also reduce the odor. Various masking agents or fragrances may also be used to mask the odor.

Of particular interest are soy products derived from soy strains that are rich in sucrose, in particular soy milk of high sucrose content and any soy product derived therefrom. Preferred for use in the compositions of the present invention are non-denatured soy products, in particular non-denatured soy products that are rich in sucrose. These are preferably decontaminated as described in EP-1236465, for example by gamma irradiation of non-denatured soymilk powder, preferably at a dose of about 10 kGy.

The compositions of the invention further contain at least a topically acceptable organic salt of a metal selected from the group consisting of magnesium, zinc and copper. Topically acceptable salt forms of magnesium, zinc and copper are those that are biologically acceptable for the skin and/or mucous membranes. These salts in particular will lack irritation and show a sufficiently low level of toxicity. The acids from which these salts are derived are organic acids, which will be selected such that they fulfill these requirements.

Particular organic salts of magnesium, zinc and copper for use in the compositions of the present invention are organic acids such as, for example, acetic acid, ascorbic acid, benzoic acid, carbonic acid, citric acid, lactic acid, lauric acid, pidolic acid, propanoic acid, myristic acid, palmitic acid, salicylic acid, stearic acid, tartaric acid, malic acid and the like acids. Other organic acids are derived from naturally occurring amino acids such as, for example, aspartic acid. Other particular salts are derived from mono- or polyhydroxyalkyl carboxylic acids. As used herein mono- or polyhydroxyalkyl carbonic acids refers to carbonic acids having an alkyl chain being mono- or polysubstituted with hydroxy groups with a maximum of one hydroxy group per carbon atom, and wherein the said carbonic acids have from 2 to 7 carbon atoms. A preferred subgroup of said polysubstituted hydroxyalkyl carbonic acids are those that are derived from sugars, in particular from sugars having 5 or 6 carbon atoms, more preferably gluconic acid.

The compositions of the invention preferably contain the magnesium, zinc and/or copper salts derived from aspartic acid or in particular salts derived from gluconic acid, or mixtures thereof.

In a preferred embodiment, there is provided a composition comprising at least one soy product as defined herein of formula (I) and at least one topically acceptable organic salt of a metal selected from the group consisting of magnesium, zinc and copper.

Particular zinc and magnesium salts for use in said preferred embodiment are the aspartate or gluconate salts. Most preferred are magnesium aspartate, copper gluconate and zinc gluconate.

In a more preferred embodiment, there is provided a composition comprising at least one soy product and at least one topically acceptable organic salt of zinc, at least one topically acceptable organic salt of magnesium and at least one topically acceptable salt of copper. Particular zinc, magnesium and copper salts for use in said more preferred embodiment are the aspartate or gluconate salts.

Particularly preferred embodiments are those compositions that contain one or more salts selected from magnesium aspartate, zinc gluconate and copper gluconate.

The organic metal salts used in the compositions of the invention are commercially available or can be prepared by methods known in the art.

If the salts are of magnesium, the w/w ratio of the magnesium and zinc salts is in the range of about 2:1 to 1:2, preferably from about 1.5:1 to 1:1.5, more preferably the w/w ratio is about 1:1. If present, the copper salts will be present in an amount equal or, which is preferred, lower than that of the magnesium and/or zinc salts. The w/w ratio of the copper salts to the magnesium and/or zinc salts may be in the range of about 1:1 to 1:20, preferably from about 1:5 to 1:15, more preferably said w/w ratio is about 1:10.

A particularly preferred organic salt mixture is the mixture containing magnesium aspartate, zinc gluconate and copper gluconate. Such a mixture is commercially available under the trade name Sepitonic™ M3, available from the company Seppic. Sepitonic™ M3 is an aqueous solution containing 4.75% of magnesium aspartate, 4.75% of zinc gluconate, 0.5% of copper gluconate, water and some preservative.

The said organic salts preferably are formulated in an aqueous medium, optionally in the presence of water-soluble organic components such as mono- or polyalkanols, in particular polyalkanols having emollient properties such as glycerine.

The compositions of this invention preferably contain a stabilizing system. The latter may for example comprise one or more components selected from the group consisting of one or more antioxidants, chelating agents and preservatives.

The compositions of this invention may contain one or more preservatives. Preservatives are useful for substantially preventing microbial decomposition. Examples of preservatives include phenoxyethanol and parabens such as methyl-paraben, ethylparaben, and propylparaben. Other examples of preservatives are listed on pages 1654-55 of the International Cosmetic Ingredient Dictionary and Handbook, eds. Wenninger and McEwen (CTFA, 7th ed., 1997), hereinafter referred to as the “Cosmetic Handbook.” The compositions may comprise from about 0.01% to about 20%, by weight (more preferably, from about 0.5% to about 5%, by weight) of preservative. Microbial contamination can also be eliminated by gamma irradiation or microfiltration, or by brief heat treatments that do not result in the elimination of protease inhibitory activity.

Antioxidants and/or chelating agents may also be used to increase shelf life and stability of the compositions. Antioxidants may be added both for formulation stabilization and for biological efficacy. Antioxidant compounds and their derivatives include, but are not limited to, water-soluble antioxidants such as sulfhydryl compounds and their derivatives (e.g., sodium metabisulfite and N-acetyl-cystein), lipoic acid and dihydrolipoic acid, resveratrol, acetyl-cysteine (Ineferine™) or lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, propolis, and legume extracts. Other examples of antioxidants may be found on pages 1612-13 of the Cosmetic Handbook. The compositions of the present invention may comprises the antioxidant in an amount of from about 0.001% to about 20%, by weight (e.g., from about 0.01% to about 10% by weight) of the composition.

Chelating agents are also useful in assisting the stabilization of the compositions of this invention. Examples of chelating agents include EDTA and derivatives thereof (e.g., disodium EDTA and dipotassium EDTA), Iniferine™, lactoferrin, and citric acid. Other examples of chelating agents are listed on page 1626 of the Cosmetic Handbook.

The compositions of the present invention may comprise the chelating agent in an amount of from about 0.001% to about 20%, by weight (e.g., from about 0. 01% to about 10% by weight) of the composition.

Thickening agents (e.g., thickeners or viscosity enhancing agents) may be utilized in the compositions of this invention to alter their viscosity. The desired viscosity of the composition will depend upon the intended use (e.g., as a bath product, cream, lotion, or gel). For example, in applications such as bath or wash products, the viscosity of the composition should be relatively low, similar to an aqueous solution. Application as a cream, lotion, or gel will have slightly higher viscosity (e.g., between about 100 cPs and 100,000 cPs).

Thickening agents that can be added to the compositions of this invention to alter viscosity include polymers such as polyacrylates (e.g., polyacrylamide). Other examples of viscosity modifying agents are listed on pages 1692-97 of the Cosmetic Handbook. To achieve the appropriate viscosity, compositions of the present invention may comprise from about 0.01% to about 20%, by weight (e.g., from about 0.1% to about 5%, by weight) of a thickening agent.

The compositions of the invention are prepared by adding the metal salt or mixture of metal salts to the soy component or vice versa. Preferably the metal salt or metal salts are in an aqueous formulation. The compositions of the present invention therefore are mostly of aqueous nature.

In the compositions of the present invention the w/w ratio of the soy product to the said organic metal salts may vary but in particular is in the range of about 50:1 to 1:1, further in particular from 30:1 to 1:1, still further in particular from 20:1 to 2:1, preferably from about 20:1 to 5:1, more preferably the w/w ratio is in the range of about 20:1 to 10:1. These w/w ratios relate to the total amount of dry soy product and dry salts. Hence if the salts are used in solution, e.g. when using the Sepitonic™ product mentioned above, the quantity of salts need to be recalculated in function of their dilution. This is in the particular case where Sepitonic™ is used, where in that instance the range soy product: Sepitonic™ for example for the first range is 50:10.

This invention further relates to topical formulations containing a composition as defined herein. Topical compositions comprise as well dermatological formulations (or topical pharmaceutical formulations), and cosmetical formulations. Said topical formulations may further contain other ingredients or additives used in dermatological or in cosmetic formulations, including other active ingredients. Examples of further ingredients or additives are surfactants, emulsifiers, consistency factors, conditioners, emollients, skin caring ingredients, moisturizers, thickeners, lubricants, fillers, binding agents, anti-oxidants, preservatives, active ingredients, in particular dermatologically active ingredients, fragrances and the like. Active ingredients as mentioned herein comprise, for example, anti-inflammatories, anti-bacterials, anti-fungals and the like agents. Of particular interest are any active ingredients suited for topical applications.

The topical formulations according to the invention can further include one or more of a variety of optional ingredients, such as coloring agents, opacifying agents and the like.

The soy product preferably is used in the formulations for topical application at concentrations from 0.001% to 10% and preferably from 0.1% to 5%, more preferably from 0.5 % to 2 %. The organic salts of metals taken together should be used in the formulations for topical application between about 0.001% to 1% and preferably from about 0.01 to 0.2%, more preferably from about 0.05 % to 0.1 %.

Unless indicated otherwise, all percentages in the preceding and following paragraphs are w/w percentages.

The formulations according to the present invention can be prepared by adding the appropriate ingredients to a composition of the invention, or vice versa by adding the composition to an appropriate cosmetic or dermatological formulation base. It is also possible to mix all the ingredients individually, i.e. without making a separate composition as defined herein.

The formulations according to the present invention can be prepared by mixing the appropriate ingredients. It is also possible to make premixes and to add ingredients or other premixes. In a preferred method of preparation, the compositions of this invention are emulsion-based, in particular oil-in-water emulsions and are made by preparing an aqueous phase containing all hydrophilic components, an oil phase containing all lipophilic components, and subsequently adding the oil phase to the aqueous phase as to prepare an emulsion. Preferably, a premix of the soy product in some water is added after the formation of the emulsion.

The compositions and topical formulations subject of the present invention are useful to combat or to treat the effects of skin aging. The effects of skin aging comprise those associated with the aging of the skin such as the appearance of fine lines, fine wrinkling, wrinkling, loss of skin firmness, skin tightening and suppleness. The effects of skin aging are due to aging as such, but also to aging of the skin caused by external factors such as exposure to environmental factors such as exposure to sunlight, wind, atmospheric poluants and the like, or a combination of these factors.

Soy products and in particular any non-denatured soy products as mentioned herein, are known to have a number of beneficial effects such as increasing skin firmness and elasticity, even tone and texture to inhibit skin pigment formation, to combat and treat the effects of skin aging and to stimulate the proliferation of dermal cells and in particular the proliferation of fibroblasts, to increase glycoaminoglycan and stimulate collagen formation, to prevent and treat sun-induced damage and acne. Additionally, they have been found to be effective as hair growth retardants. These effects and properties are even more present when the soy products are non-denatured soy products. Certain organic salts of copper, zinc and magnesium have also beneficial effects on the appearance of the skin.

It now has been found that in the compositions and formulations of this invention, the soy products mentioned in this specification and claims and the organic salts of zinc, magnesium and/or copper, in particular the salts specified herein, act synergistically on a number of the beneficial properties of these components. Similar synergistic effects may be present in mixtures containing the organic salts of two or three of the metals specified herein. These synergistic effects are especially present in the instance where use is made of a mixture of soy products and zinc gluconate, magnesium aspartate and copper gluconate.

The topical formulations according to the invention may be in the form of a solution, a hydrophilic lotion, an ointment, a cream or a gel. The formulations may also be, for example, in the form of oil-in-water, water-in-oil or multiple emulsions, foaming products or in liposome form.

Preferred formulations are gel and cream based.

All the topical formulations as described above can be applied on the skin by means of wipes.

The topical formulations of the invention may be applied in the morning and/or evening. They may be applied on those parts of the body where skin aging is prominent, i.e. on the face, the body or the hands.

The following examples are meant to illustrate the invention and not to limit it thereto.

### EXAMPLES

### Example 1

In the following formulation examples all percentages are by weight (w/w).

The formulations are prepared in the following manner. First an aqueous phase is made which is heated. Subsequently the gellyfying agents are added.

The oily components are melted and mixed into an oily phase, which is emulsified in the water phase. The thus formed emulsion is allowed to cool and the active ingredients are added at a temperature of 35° C. Then the fragrance is added at a temperature of 30° C.

High sucrose soy milk powder is an ingredient obtained from soy milk with high sucrose content as described in EP-1236465.

Formula 1

| INCI NAME | |
|---|---|
| % of Active Ingredient | [%] w/w |
| Aqua | 59.979 |
| Acrylates/C10-30 Alkyl Acrylate Crosspolymer | 0.200 |
| PEG-20 Methyl Glucose Sesquistearate | 4.450 |
| Glycerin | 2.000 |
| Methylparaben | 0.400 |
| Methyl Glucose Sesquistearate | 1.550 |
| Cetearyl Ethylhexanoate(90%), Isopropyl Myristate(10%) | 17.500 |
| Olus 87%/Hydrogenated Vegetable Oil 10%/Candelilla | 0.500 |
| Cera 3% | |
| Propylparaben | 0.200 |
| Butyrospermum Parkii | 1.000 |
| Cetyl Alcohol | 1.000 |
| Sodium Hydroxide | 0.021 |
| Aqua | 0.200 |
| High Sucrose Soymilk Powder | 1.000 |
| Phenoxyethanol | 0.800 |
| PEG-8 | 2.000 |
| Butylene Glycol | 3.000 |
| Lactoferrin 1%/Glycerin 5%/Methylparaben 0.3%/ | 1.000 |
| Potassium Sorbate 0.15%/Aqua 93.55% | |
| Zinc Gluconate (4.75%)/Magnesium Aspartate (4.75%)/ | 1.000 |
| Copper Gluconate (0.5%)/Aqua (89%)/Phenoxyethanol (1%) | |
| Chlorhexidine Digluconate 20%/Aqua 80% | 0.250 |
| Sodium Benzoate | 0.200 |
| Polyacrylamide (40%)/C13-14 Isoparafin (20%)/ | 1.500 |
| Laureth-7 (5.5%)/Aqua (34.5%) | |
| Perfume | 0.250 |
| TOTAL | 100.0% |

Formula 2

| INCI NAME | |
|---|---|
| % of Active Ingredient | [%] w/w |
| Aqua | 44.680 |
| Panthenol 75%/Aqua 25% | 0.500 |
| Aqua (32%), Glyceryl Polymethacrylate (67%), Propylene | 4.000 |
| Glycol (1%) | |
| Glycerin | 4.000 |
| Lactose | 4.000 |
| C12-15 Alkyl Benzoate | 4.000 |
| Dimethicone | 2.000 |
| Arachidyl Alcohol (55%)/Behenyl Alcohol (30%)/ | 2.000 |
| Arachidyl Glucoside (15%) | |
| Cetearyl Alcohol (80%)/Cetearyl Glucoside (20%) | 3.500 |
| Disodium EDTA | 0.200 |
| BHT | 0.070 |
| Bis-Phenylpropyl Dimethicone | 3.000 |
| Polyacrylamide (40%)/C13-14 Isoparaffin (20%)/ | 1.000 |
| Laureth-7 (5.5%)/Aqua (34.5%) | |
| Aqua | 19.100 |
| Glycine Soja | 2.000 |
| High Sucrose Soymilk Powder | |
| Sodium Benzoate | 0.200 |
| Parfum | 0.500 |
| Phenoxyethanol (72.5%)/Methylparaben (15.5%)/ | 1.000 |
| Ethylparaben (4%)/Propylparaben (2%)/Butylparaben (4%)/ | |
| Isobutylparaben (2%) | |
| Benzyl Alcohol | 0.500 |
| Zinc Gluconate (4.75%)/Magnesium Aspartate (4.75%)/ | 1.000 |
| Copper Gluconate (0.5%)/Aqua (89%)/ Phenoxyethanol (1%) | |
| Lactoferrin (1%)/Glycerin (5%)/Methylparaben (0.3%)/ | 1.000 |
| Potassium Sorbate (0.15%)/Aqua (93.55%) | |
| Aqua | 0.400 |
| Citric Acid | 0.100 |
| Mica | 1.000 |
| Colorant | 0.250 |
| TOTAL | 100.0% |

1. A composition comprising at least one soy product and at least one topically acceptable organic salt of a metal selected from the group consisting of zinc, magnesium and copper.

2. A composition according to claim 1 wherein the skin acceptable non-toxic organic salts of zinc are salts derived from naturally occurring amino acids, from hydroxyalkyl acids, the latter in particular being sugar-derived acids, or other cosmetically acceptable acids.

3. A composition according to claim 2 wherein the organic salts are the salts of gluconic acid or aspartic acid.

4. A composition according to any of claims 1-3, wherein the soy product is a non-denatured soy product.

5. A composition according to any of claims 1-4, wherein the w/w ratio of the soy product to the said organic metal salts may vary but in particular is in the range of about 50:1 to 1:1, further in particular from 30:1 to 1:1, still further in particular from 20:1 to 2:1, preferably from about 20:1 to 5:1, more preferably the w/w ratio is in the range of about 20:1 to 10:1 and wherein the w/w ratios relate to the total amount of dry soy product and dry salts.

6. A topical formulation comprising a composition as claimed in claims 1-5 and further ingredients.

7. A formulation according to claim 6, wherein the soy product is used at concentrations from 0.001% to 10% and preferably from 0.1% to 5%, more preferably 30 from0.5%to2%.

8. A formulation according to claim 6 wherein the organic salts of metals taken together are present at concentrations from about 0.001% to 1% and preferably from about 0.01 to 0.2%, more preferably from about 0.05 % to 0.1 %.

9. Use of a composition as claimed in claims 1-5 for manufacturing a topical or in particular a cosmetic formulation.

10. Use of a composition as claimed in claims 1-5 or of a formulation as claimed in claims 6-8 for increasing skin firmness and elasticity, even tone and texture, to combat and treat the effects of skin aging, to prevent and treat sun-induced damage and acne.
